(19) United States
(12) Patent Application Publication
(10) Pub. No.: US 2021/0145638 A1
(43) Pub. Date: May 20, 2021

(54) SURGICAL INSTRUMENT, IN PARTICULAR PHACOEMULSIFICATION HANDPIECE

(71) Applicant: WEFIS GmbH, Koln (DE)

(72) Inventors: Dietrich WERNZ, Koln (DE); Stefan WORSDORFER, Koln (DE)

(21) Appl. No.: 17/044,477

(22) PCT Filed: Apr. 3, 2019
(86) PCT No.: PCT/EP2019/058353

§ 371 Date: Oct. 1, 2020

(30) Foreign Application Priority Data
Apr. 4, 2018 (EP) PCT/EP2018/058600

(51) Int. Cl. A61F 9/007; A61F 9/00

(52) U.S. Cl. CPC A61F 9/00745; A61F 9/0008

(57) ABSTRACT

The surgical instrument (10), in particular a phacoemulsification handpiece, is provided with a housing (12), an ultrasonic transducer (40) positioned in the housing (12), and a tool (46) which is operatively connected to the ultrasonic transducer (40) for the purpose of ultrasonic excitation. The ultrasonic transducer (40) has a sonotrode (56), a resonator (58) and a piezoelectric ceramic element (60) positioned therebetween. The sonotrode (56) and/or the resonator (58) has an aluminum alloy or consists of an aluminum alloy having a tensile strength greater than 400 N/mm². The housing (12) can consist of plastic and has an inner housing part (16) made of plastic, in which the ultrasonic transducer (40) is positioned. Cooling openings (82) are formed in the plastic inner housing part (16) through which openings a rinsing fluid for cooling the sonotrode (56) or the ultrasonic transducer (40) flows, said fluid passing along between the outer housing part (14) and the inner housing part (16).

The invention relates to a surgical instrument, in particular a disposable instrument and preferably a phacoemulsification handpiece, which in particular is disposable.

Phacoemulsification handpieces are used as surgical instruments in ophthalmic surgery. Examples of such surgical instruments are described in WO-A-93/15703-A, WO-A-00/53136-A, EP-A-2 011 458, DE-A-102 09 495 and DE-C-4 008 594.

WO-A-89/06515 describes a device for in vivo angioplasty comprising a sonotrode made of an aluminum alloy which may be Al-2024 or Al-7075.

Medical ultrasound devices with waveguides of different materials, such as e.g. titanium, iron or steel, are known from WO-A-2997/070081.

Further medical ultrasound devices are known from U.S. Pat. No. 7,431,728 B.

A phacoemulsification handpiece serves to crush the eye lens and to suction off remainders of tissue. To this end, the tool is designed as a hollow needle and is caused by a vibration mechanism of the handpiece, which is configured as an ultrasonic transducer, to perform high-frequency linear reciprocating movements. A rinsing fluid, for example an emulsion fluid, is supplied around the exterior of the hollow needle, which fluid reaches the surgical site and is suctioned off from there together with dissolved tissue components through the tool and a suctioning duct of the handpiece.

The costs for manufacturing a phacoemulsification handpiece are not negligible and are essentially determined by the ultrasonic transducer and the housing. Therefore, due to the comparatively high manufacturing costs phacoemulsification handpieces are used multiple times. Thus, the individual components have to be made of comparatively durable materials. For multiple use of a phacoemulsification handpiece, it is absolutely necessary to sterilize the same after each use or prior to further use. This also entails additional costs and is problematic as well, which is why it has already been considered to develop phacoemulsification hand-pieces for single use. However, this failed until now due to the still rather high manufacturing costs.

It is an object of the invention to further simplify the individual components of a surgical instrument, in particular a phacoemulsification handpiece, to enable a more economic production.

To achieve this object the invention provides a surgical instrument, in particular a phacoemulsification handpiece which comprises
a housing
an ultrasonic transducer arranged in the housing and
a tool which is operatively connected to the ultrasonic transducer for the purpose of ultrasonic excitation
the ultrasonic transducer comprising a sonotrode, a resonator and a piezoceramic element arranged therebetween and
the sonotrode and/or the resonator comprising an aluminum alloy or being made of an aluminum alloy, wherein the aluminium alloy has a tensile strength greater than 400 N/mm².

Analogously, the invention provides that the ultrasonic transducer of the phacoemulsification handpiece is made of an aluminum alloy having a minimum tensile strength. The ultrasonic transducer comprises the sonotrode, the resonator and at least one piezoceramic element held clamped between the sonotrode and the resonator. According to the invention, aluminum alloys are now used for the sonotrode and the resonator, which have a tensile strength of more than 400 N/mm² and in particular more than 450 N/mm². Surprisingly, it has been found that such aluminum alloys are sufficiently strong to withstand the high-frequency bending waves generated when the piezoceramic element is driven, and this in particular for the duration of an operation. The coupling of the sonotrode with the tool is rigid. Furthermore, the tool itself is also made of a substantially rigid or stiff material such as metal and in particular e.g. steel, iron or titanium, so that the acoustic energy, i.e. the vibrations of the sonotrode, is/are transferred to the tool and through the same substantially without loss.

According to the invention a high-strength aluminum alloy is used as the material for the sonotrode and the resonator, in particular. Examples for such alloys which have proven suitable according to advantageous embodiments of the invention include:

AlCu4SiMg

AlCu4Mg1

ALZn4.5Mg1

AlZn5.5MgCu

AlZnMg3Cu

AlZn8MgCu.

Suitable aluminum alloys for the purposes of the invention are mentioned for example in the leaflet W2, 11th edition, “Aluminium-Knetwerkstoffe”, published by Gesamtverband der Aluminiumindustrie e.V. (GDA) ISBN 3-937171-00-2, in particular table 9, pages 54 to 60 in particular pages 55 to 60.

In a further advantageous embodiment of the invention it is provided that the sonotrode comprises a midsection arranged adjacent to the resonator, in which section the sonotrode is supported in the housing, a rear end coupled to the resonator and a front end facing away from the resonator and being reduced compared to the midsection, said end being operatively connected to the tool, and that a suction duct extends through the sonotrode from the front end thereof to the rear end thereof connected to the resonator.

Regarding the layout and the design of an ultrasonic transducer for a phacoemulsification handpiece, design rules exist that are known to a skilled person. Reference is made as an example to Dr.-Ing. E. G. Lierke, Dr.-Ing. W. Littmann, Dipl.-Ing D. Simon, Dr.-Ing. T. Hemsel: “Zur Theorie der piezoelektrischen Ultraschallverbundschwinger mit praktischen Schlussfolgerungen für den Entwicklungsingenieur”, University of Paderborn, Faculty of Mechanical Engineering, Department of Mechatronics and Dynamics, August 2010.

As already mentioned above, the invention provides for the use of aluminum alloys for the manufacture of the sonotrode. However, this does not exclude that parts of the sonotrode can be made of another metal material than said aluminum alloys.

The sonotrode and/or the resonator of the ultrasonic transducer is/are subjected to increased temperatures during operation. In the known multi-use phacoemulsification handpieces, the sonotrode and the resonator are located in a metal inner housing which is surrounded by a metal outer housing while forming an annular space. A rinsing fluid flows through the annular space and reaches the tool, as already described above. This rinsing fluid cools the metal inner housing and, via the same, thus also cools the sonotrode and/or the resonator.

If a disposable phacoemulsification handpiece is to be designed, low-cost material should be used for the housing. If, for example, plastic material were used, a problem could be that the inner housing of plastic material is by far less thermally conductive than would be the case for an inner housing of metal. Thus, a plastic inner housing should have recesses acting as cooling openings and in which the sonotrode and the resonator are exposed, respectively. However, in this case, the rinsing fluid gets in contact with the material of the ultrasonic transducer. This material should be biocompatible therewith. Thus, upon contact with the emulsion fluid, which may e.g. be a saline solution, no reaction products must be produced by this material, which products would then be transported by the rinsing fluid to the tissue to be treated and would biochemically affect the tissue. Therefore, it may be advantageous to make those components of the sonotrode or the resonator which come into contact with the rinsing fluid from another material, in particular another metal material than that of the aluminum alloys used according to the invention, if it is found that the aluminum alloy used is not biocompatible. In this respect, e.g. titanium, iron, steel or another biocompatible metal or a corresponding metal alloy are viable materials.

As an alternative, to solve the above-mentioned problem, the sonotrode and/or the resonator could be provided with a compatible coating.

In a further advantageous embodiment of the invention, it may be provided that the resonator is of a hollow cylindrical design, and that the sonotrode has a rear end mechanically coupled with the resonator and extending through the resonator, which, for biasing the piezoceramic element arranged between the resonator and the sonotrode, is in mechanical engagement, in particular threaded engagement with the resonator or is adapted to be locked therewith by clamping, latching or pinning or any other way.

For connecting the tool to the sonotrode, it is advantageous if at its front end operatively connected to the tool, the sonotrode is lead out of an opening of the housing with a clearance or play, wherein a holder element can be set, in particular plugged or screwed, onto the housing at the opening thereof, from which holder element an elastic sleeve extends that surrounds the tool on the outside. As already described above, an elastic sleeve is slipped onto the hollow needle of phacoemulsification handpieces. This elastic sleeve is located on a holder element configured as a standardized screw connection for surgical elements. Here, the holder element is screwed onto a threaded connecting piece of the housing which forms the opening of the housing. For the reasons stated above, the outer diameter of the threaded connecting piece should be normed so as to be able to use standard connections. However, in turn, this means that the front end of the sonotrode led out from the threaded connecting piece or arranged in the threaded connecting piece must have a certain undersize with respect to the inner diameter of the threaded connecting piece, so that an annular space is formed between the sonotrode and the threaded connecting piece, via which the rinsing fluid can leave the housing and get into the region between the tool and the elastic sleeve. Surprisingly, it has been found that even then the aluminum alloys to be used according to the invention are still sufficiently strong and withstand the bending waves occurring during the operation of the sonotrode, although the wall thickness of the front end of the sonotrode through which the suctioning duct extends is comparatively thin.

An aspect separate and independent from the above aspect of the invention, namely the use of special aluminum alloys having a minimum tensile strength, is the feasibility to be able to use a plastic housing for a surgical instrument, in particular for a phacoemulsification handpiece. It is readily obvious to a skilled person that, by itself, the use of plastics as the material for the housing of the surgical instrument does not necessarily require the sonotrode and/or the resonator of the surgical instrument to be made of a special aluminum alloy or of a special material at all.

Insofar, the suggestion to use plastics as the material for the housing should be seen as aspect separate from the previous aspect of the invention.

According to this second aspect the invention provides a surgical instrument, in particular a phacoemulsification handpiece, comprising
a housing
an ultrasonic transducer arranged in the housing and
a tool (comprising in particular metal such as steel, iron or titanium) which is operatively connected to the ultrasonic transducer for the purpose of ultrasonic excitation
the ultrasonic transducer comprising a sonotrode, a resonator and a piezoceramic element arranged therebetween and
wherein it is provided in particular that the housing comprises an inner housing part surrounding at least a part of the sonotrode and an outer housing between which an annular space is provided for supplying and/or for directing a rinsing fluid to the tool.

In an advantageous development of this additional aspect of the invention it may be provided that the housing comprises an inner housing part surrounding at least a part of the sonotrode and an outer housing between which an annular space is provided for supplying and/or for directing a rinsing fluid to the tool. As already mentioned above, in a plastic inner housing part, cooling the sonotrode by the rinsing fluid flowing through the annular space my be problematic. This depends, for example, on the thickness of the inner housing part, while it should be noted that the thickness of the inner housing part should not fall below a certain thickness value for reasons of stability. This may possibly give rise to the problem that the inner housing part is too thick-walled to still provide sufficient cooling for the sonotrode through the rinsing fluid flowing around the inner housing part.

As such, it is advantageous if the inner housing part has cooling openings within which the sonotrode and in particular the midsection of the sonotrode is exposed and is in contact with rinsing fluid for the purpose of cooling.

If the sonotrode were now made of an aluminum material, as suggested by the first aspect of the invention, the problem of a possibly no longer existing biocompatibility would arise. Therefore, care would have to be taken, e.g. by means of a corresponding coating that the aluminum material does not get in contact with the rinsing fluid at least in those regions of the sonotrode in which the same is exposed in the cooling openings.

In a further advantageous embodiment of the invention it may be provided that the inner housing part has an exit opening for the front end of the sonotrode, and that the outer housing part has an exit opening aligned with the exit opening of the inner housing part and spaced therefrom in the longitudinal direction of the sonotrode, in which outer housing part exit opening the front end of the sonotrode is arranged and/or beyond which the front end of the sonotrode protrudes and on which a holder element can be set, in particular plugged or screwed, from which an elastic sleeve extends which surrounds the tool on the outer side. Thus, the outer housing part protrudes beyond the inner housing part at the front end of the surgical instrument. The sonotrode protrudes from the inner housing part through the exit opening of the inner housing part, in particular by its reduced front end. This front end then extends further through the outer housing part and through the exit opening thereof which is formed in particular as a connecting piece, or the front end ends in this exit opening. Thus, the annular space between the two housing is adjoined by a further annular space which is defined by the outer housing part and the sonotrode or the front end thereof. Now, the rinsing fluid contacts the sonotrode in this region as well. Thus, the material of the sonotrode should be biocompatible. If an aluminum alloy is used as the material, as suggested by the first aspect of the invention, and if it were found that this material is not biocompatible, the sonotrode would e.g. have to be coated with a biocompatible material in this region. As an alternative, the part of the sonotrode extending through the annular space upstream of the inner housing part could of course be made of another metal material than aluminum or an aluminum alloy. As it were, a sonotrode would be obtained which is made of different metal materials. The part of the sonotrode that is not made of aluminum or an aluminum alloy may be made e.g. of titanium, iron or steel or another biocompatible metal that withstands the bending waves occurring during the operation of the ultrasonic transducer.

In a further advantageous embodiment of the invention it may be provided that a further annular space adjoins the annular space between the inner housing part and the outer housing part at the exit opening of the inner housing part, which further annular space is formed by the outer housing part and the front end of the sonotrode and which extends into the gap defined by the tolerance between the front end of the sonotrode and the exit opening of the outer housing part and, from there, transitions into a gap between the tool and the elastic sleeve.

As already described above, the ultrasonic transducer is arranged and supported in the housing of the surgical instrument. In this respect it is feasible if the sonotrode has two axially spaced receiving grooves for a respective bearing ring, in particular for an elastic bearing ring and preferably for an O-ring, the two bearing rings abutting on the housing, in particular the inner housing part, from the inside for the purpose of supporting the ultrasonic transducer.

As an alternative to the above-mentioned support and centering of the sonotrode in the housing or in the inner housing part, it may be provided that the sonotrode has a receiving groove for a bearing ring, in particular an elastic bearing ring and preferably for an O-ring, at its rear end opposite the front end and that the front end of the sonotrode is centered by inward protruding ribs or the like protrusions of a connecting piece of the housing.

If the inner housing part of the surgical instrument has cooling openings, it is advantageous if the cooling openings are arranged in the region of the housing or the inner housing part between the two bearing rings, the two receiving grooves being formed in the midsection of the sonotrode. The bearing rings thus seal the region on either side of the array of cooling openings. Thus, they not only serve for supporting purposes, but also prevent the ingress of rinsing fluid into the inner housing.

It is further feasible that the housing comprises an inlet opening for the supply of rinsing fluid, opening into the annular space.

It may be advantageous if at the front end of the sonotrode opposite the rear end, the housing has a passage opening for a suctioning hose which is connected to the suctioning duct extending through the sonotrode.

In a further advantageous embodiment of the invention a connection cable may be led out from the housing, which is connected to the piezoceramic element.

The invention will be explained hereunder in more detail with reference to two embodiments and to the drawings. The Figures show:

FIG. 1 a longitudinal section through a phacoemulsification handpiece,

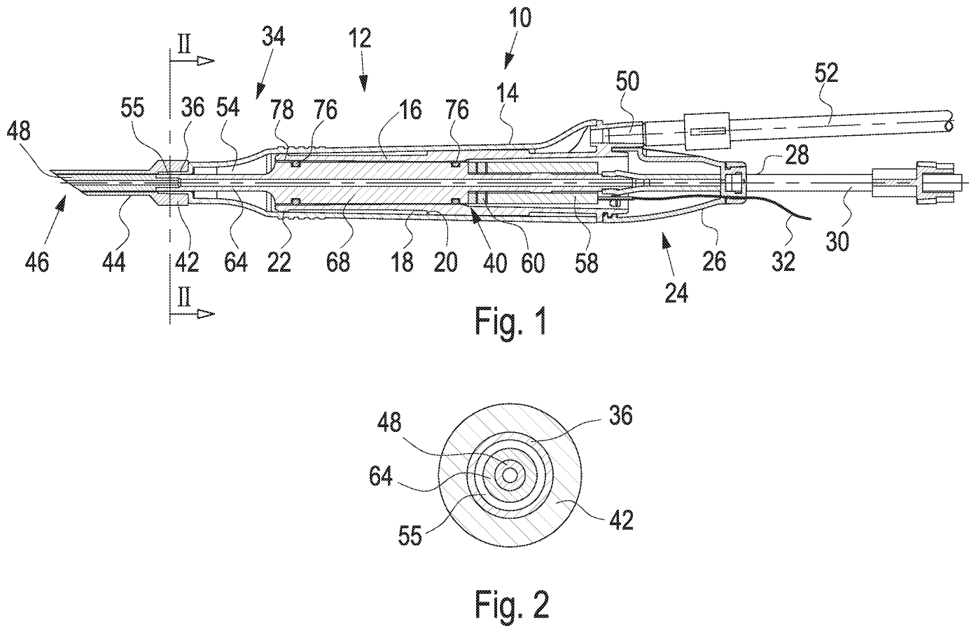

FIG. 2 a cross section along line II-II in FIG. 1,

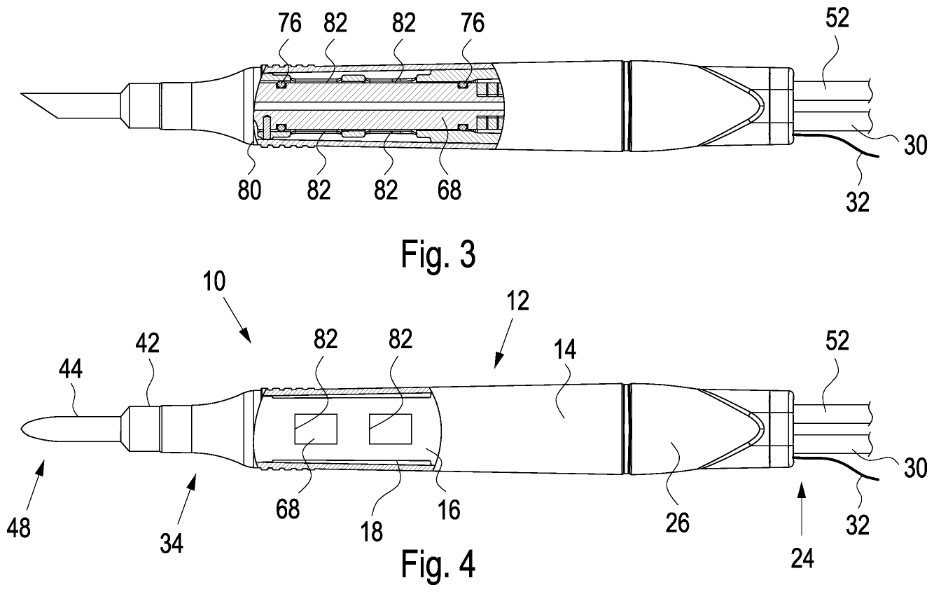

FIG. 3 a first side view on the phacoemulsification handpiece of FIG. 1 with the housing partially broken away and shown in section,

FIG. 4 another side view of the phacoemulsification handpiece with the inner housing part also partially broken away, but shown in side view,

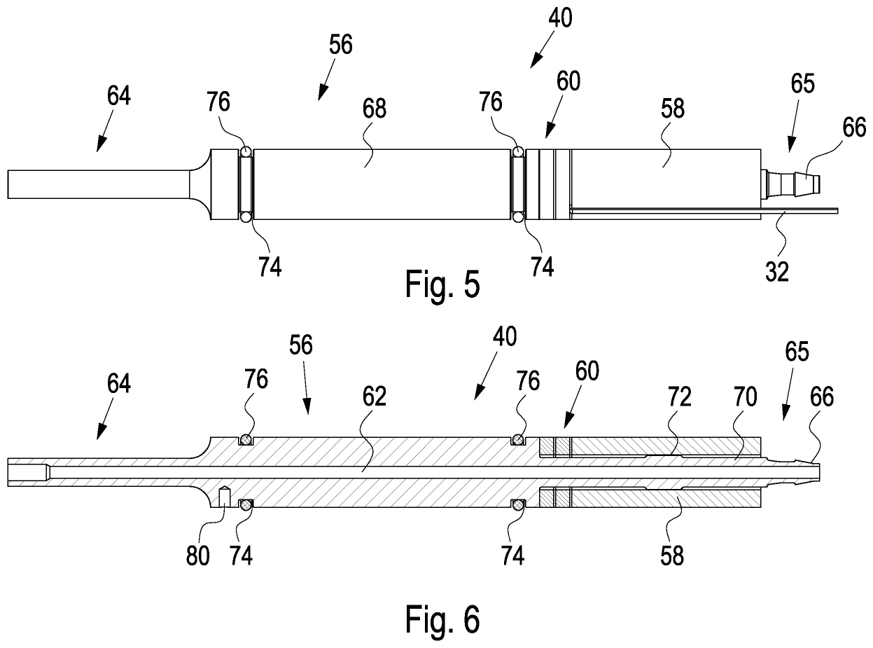

FIG. 5 a side view of the ultrasonic transducer of the phacoemulsification handpiece,

FIG. 6 a longitudinal section through the ultrasonic transducer of FIG. 5,

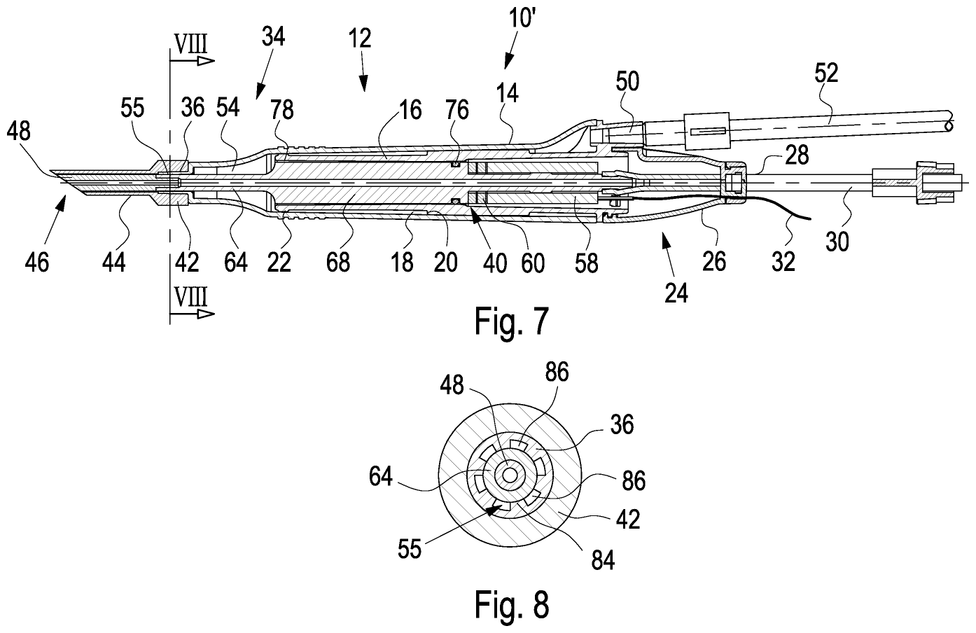

FIG. 7 a longitudinal section through an alternative embodiment phacoemulsification handpiece and

FIG. 8 a cross section along line VIII-VIII in FIG. 7.

FIGS. 1 to 4 are different illustrations of a phacoemulsification handpiece 10 according to a first embodiment of the invention. The handpiece 10 comprises a housing 12 of plastic material. This housing has an outer housing part 14 and an inner housing part 16 arranged in the outer housing part 14. A first annular space 18 is formed between the two housing parts 14, 16, the inner housing part 16 being centrally supported relative to said annular space by a plurality of outer protrusions 20, 22. At the rear end 24 of the housing 12 the same closed by a single- or multi-part cover element 26 from which a suctioning hose is led out through a passage opening 28. Further, a connection cable 32 is led out from the cover element 26. These two components will be addressed further below.

As can be seen from FIGS. 1 to 4, the outer housing part 14 protrudes beyond the inner housing part 16 at the front end 34 of the housing 12. A connecting piece 36 is situated at the front end 34 of the housing 12, which connecting piece forms an exit opening 38 for an ultrasonic transducer 40 arranged in the housing 12. A holder element 42 is screwed onto the connecting piece 35, with an elastic sleeve 44 being located on the holder element, the sleeve surrounding a tool 46 in the form of a hollow needle 48 (with a lumen 49) of the handpiece 10.

Rinsing fluid flows into the annular space 18 of the housing via an inlet opening 50 to which a rinsing fluid hose 52 is connected, the fluid flowing from the annular space 18 into another annular space 54 and from there into the gap between the tool 46 and the elastic sleeve 44 via the exit opening 38.

The ultrasonic transducer 40 of the phacoemulsification handpiece 10, already addressed above, is located in the inner housing part 16 of the housing 12.

As can be seen in FIGS. 5 and 6, the ultrasonic transducer 40 comprises a sonotrode 56, a resonator 58 and at least one piezoceramic element 60. The connection cable 32 is connected to the piezoceramic element 50. A suction duct 62 extends through the sonotrode 56, which duct is in fluid connection with the lumen of the hollow needle 48 at the front end 64 of the sonotrode 56 and whose rear end 65 is provided with a connecting piece 66 for connecting the suction hose 30. The sonotrode 56 has a midsection 68 relative to which the front end 64 is reduced. The rinsing fluid flows through a gap 55 between the connecting piece 36 and the front end 64 of the sonotrode 50 (see FIG. 2) which is in communication with the tool 46, and flows further between the tool 46 and the elastic sleeve 44 which will widen correspondingly when the tool 46 is surrounded by rinsing fluid.

Opposite the front end 44 the sonotrode 56 has a rear endpiece 70 which is also reduced and which extends through the resonator 58 designed as a hollow cylinder with which it is in threaded engagement at 72. In this manner, the at least one piezoceramic element 60 can be held clamped between the resonator 58 and the midsection 68 of the sonotrode 56. The midsection 68 of the sonotrode is provided with two circumferential receiving grooves 74, each for one elastic bearing ring 76 via which the sonotrode 56 and thus the ultrasonic transducer 40 are supported in the inner housing part 16 of the housing 12.

The distinctive feature of the sonotrode 56 described herein is to be seen in the choice of its material. According to the invention a high-strength aluminum alloy is used which has a tensile strength greater than 400 N/mm² and in particular greater than 450 N/mm².

As can be seen in FIG. 1, the inner housing part 16 surrounds the midsection 68 of the sonotrode 56, as well as the piezoceramic element 60 and the resonator 58. In the region of the transition from the midsection 68 to the reduced front end 64 the sonotrode 56 protrudes from the inner housing part 16. The exit opening 78 provided in the inner housing part 16 is adjoined by the further annular space 54 which is defined by the outer housing part 14 and the reduced front end 64 of the sonotrode 56.

According to another aspect of the invention the housing 12 is a housing of plastic material. The sonotrode 56 is supported in the plastic inner housing part 16 via the two bearing rings 76. It is indicated at 80 in FIGS. 3 and 6 that the ultrasonic transducer 40 is pinned to the plastic inner housing part 16 in the region of its sonotrode 56. During operation of the ultrasonic transducer 40, the sonotrode 56 heats up noticeably, which is why it should be cooled. This is effected by the rinsing fluid, for which purpose the plastic inner housing part 16 is provided with a plurality of cooling openings 82 in the region around the midsection 68 of the sonotrode 56 (see FIGS. 3 and 4). Within these cooling openings 82, the midsection 68 the sonotrode 56 is exposed, so that the rinsing fluid can now flow directly along the sonotrode 56 in these regions, whereby the cooling effect is much more effective than in the case of a continuous design of the plastic inner housing part 16. Due to the fact that the rinsing fluid, which gets in contact with the tissue to be treated during an operation, flows along the sonotrode 56 in the midsection 68 thereof and in the reduced end 64 thereof (i.e. in the annular space 54), care should be taken that the material of the sonotrode is biocompatible. With an aluminum alloy and a rinsing fluid which is provided as a saline solution, this is possibly not given to a sufficient degree. Therefore, the sonotrode 56 should be provided with a biocompatible coating at least in the regions mentioned before. As an alternative, also the entire front end 64 of the sonotrode 56 can consist of a biocompatible metal such as titanium. This part of the sonotrode 56 would then be mechanically connected to the remainder of the sonotrode 56 e.g. by screwing.

As can be seen in FIG. 3, the bearing rings 76 are situated on either side of the array of cooling openings 83 and thus seal the sonotrode 56 in particular from the resonator 58 and the piezoceramic element 60, so that no rinsing fluid can reach this region.

As already mentioned above, the features of the invention and of the above-described embodiment, as far as they relate to the plastic housing, are an aspect of the invention that is independent of the other aspect of the invention, i.e. the use of special aluminum alloys for an ultrasonic transducer. The features of the plastic housing and individual variants thereof are listed in the following feature groups which may be combined with each other, but are also valid by themselves and define the invention and/or embodiments of the invention in all instances.

Referring to FIGS. 7 and 88, an alternative embodiment of the centric support of the sonotrode of in the housing of the handpiece will be addressed, which bears the reference numeral 10′ in these Figures. As far as the individual elements of the handpiece 10′ of FIGS. 7 and 8 correspond in structure or function to those of the handpiece 10 of FIGS. 1 to 6, they are identified in FIGS. 7 and 8 by the same reference numerals as in FIGS. 1 to 6.

The difference between both handpieces 10 and 10′ is to be seen in the support of the sonotrode 56. In the handpiece 10′, the sonotrode 56 is supported at its rear end 65 or in its midsection by the O-ring 76, whereas its front end 84 is centered at inward protruding ribs 84 of the connecting piece 36 of the outer housing part 14 (see FIG. 8). The ribs 84 protrude into the gap 55 and divide the same into individual ducts 86. Moreover, the ribs 84 stiffen the connecting piece 36 and thus the housing. The front receiving groove 74 of the sonotrode 56, which exists in the handpiece 10 of FIGS. 1 to 6, can be omitted which is advantageous in terms of production and assembly (the front O-ring and the assembly thereof to the sonotrode are omitted).

Individual embodiments of the invention can comprise one or a plurality of the following feature groups or individual or a plurality of one or a plurality of the following feature groups.
Surgical instrument, in particular a phacoemulsification handpiece, comprising
   a housing 12
   an ultrasonic transducer 40 arranged in the housing 12 and
   a tool 46 which is operatively connected to the ultrasonic transducer 40 for the purpose of ultrasonic excitation,
   the ultrasonic transducer 40 comprising a sonotrode 56, a resonator 58 and a piezoceramic element 60 arranged between these and
   the housing 12 comprising plastic material and//or consisting of plastic material.
Surgical instrument, in which the housing 12 comprises an inner housing part 16 surrounding at least a part of the sonotrode 56 and an outer housing part 14 between which an annular space 18 is provided for supplying and/or for directing rinsing fluid to the tool 46.
Surgical instrument, in which the inner housing part 16 comprises cooling openings 82 within which the sonotrode 56 and in particular the midsection 68 of the sonotrode 56 is exposed and comes in contact with rinsing fluid for the purpose of cooling.
Surgical instrument, in which the inner housing part 16 comprises an exit opening 78 for the front end 34 of the sonotrode 56, and the outer housing part 14 comprises an exit opening 78 aligned with the exit opening 38 of the inner housing part 16 and spaced from the same in the longitudinal direction of the sonotrode 56, in which exit opening the front end 34 of the sonotrode 56 is arranged and/or beyond which the front end 36 of the sonotrode 56 protrudes and on which a holder element 42 can be set, in particular plugged or screwed, from which an elastic sleeve 44 extends that surrounds the tool 46 on the outer side.
Surgical instrument, in which the annular space 18 between the inner housing part 16 and the outer housing part 14 is adjoined at the exit opening 78 of the inner housing part 16 by a further annular space 54 which is formed by the outer housing part 14 and the front end 34 of the sonotrode 56 and which extends into a gap 55 defined by a tolerance between the front end 34 of the sonotrode 56 and the exit opening 38 of the outer housing part 14 and, from there, transitions into a gap between the tool 46 and the elastic sleeve 44.
Surgical instrument, in which the sonotrode 56 comprises two axially spaced receiving grooves, each for one bearing ring, in particular for an elastic bearing ring and preferably an O-Ring, both bearing rings abutting on the housing 12 from inside and in particular on the inner housing part 16 for the purpose of supporting the ultrasonic transducer 40.
Surgical instrument, in which the sonotrode 56 comprises a receiving groove 74 for a bearing ring 76, in particular an elastic bearing ring and preferably an O-ring, at its rear end 65 opposite the front end 64 or in the central portion, and wherein the front end 64 of the sonotrode 56 is centered by inward protruding ribs 84 or the like protrusions of a connecting piece 36 of the housing 12.
Surgical instrument, in which the cooling openings 82 are arranged in the region of the housing 12 or the inner housing part 16 situated between the two bearing rings and the two receiving grooves 74 are formed in the midsection 68 of the sonotrode 56.
Surgical instrument, in which the housing 12 comprises an inlet opening 50 in fluid communication with the annular space 18 for the supply of rinsing fluid.
Surgical instrument, in which the housing 12 has a passage opening 28 for a suctioning hose 30 at its rear end 24 opposite the front end 36 of the sonotrode 56, which hose is connected to the suctioning duct 62 extending through the sonotrode 56.
Surgical instrument, in which a connection cable 32 connected to the piezoceramic element 60 is led out from the housing 12.

### LIST OF REFERENCE NUMERALS

10 (phacoemulsification) handpiece
10′ (phacoemulsification) handpiece
12 housing
14 outer housing part
16 inner housing part
18 annular space
20 outer protrusions
22 outer protrusions
24 (rear) end of the housing
26 cover part
28 passage opening
30 suctioning hose
32 connection cable
34 (front) end of the housing
36 connection piece
38 exit opening for the sonotrode at the outer housing part
40 ultrasonic transducer
42 holder element
44 elastic sleeve
46 tool
48 hollow needle
49 lumen of the hollow needle
50 inlet opening
52 rinsing fluid hose
54 annular space
55 gap between the connecting piece and the front end of the sonotrode
56 sonotrode
58 resonator
60 piezoceramic element
62 suctioning duct
64 frontend of the sonotrode
65 rear end of the sonotrode
66 connection piece
68 midsection
70 endpiece
72 threaded engagement between the sonotrode and the resonator
74 circumferential receiving grooves
76 bearing ring
78 exit opening for the sonotrode at he inner housing part
80 pinning
82 cooling openings
84 (guiding and stiffening) ribs
86 ducts

1-19. (canceled)

20. A surgical instrument, in particular a phacoemulsification handpiece, comprising
a housing,
an ultrasonic transducer arranged in the housing and
a tool which is operatively connected to the ultrasonic transducer for the purpose of ultrasonic excitation,
the ultrasonic transducer comprising a sonotrode, a resonator and a piezoceramic element arranged therebetween and
the sonotrode and/or the resonator comprising an aluminum alloy or being made of an aluminum alloy, wherein the aluminum alloy has a tensile strength greater than 400 N/mm².

21. The surgical instrument according to claim 20, wherein the aluminum alloy is a high-strength aluminum alloy.

22. The surgical instrument according to claim 20, wherein the aluminum alloy is an alloy selected from the group of the following aluminum alloys: Al—Cu4SiMg; AlCu4Mg1; AlZn4.5Mg1; AlZn5.5MgCu; AlZnMg3Cu; AlZn8MgCu.

23. The surgical instrument according to claim 20, wherein the sonotrode comprises a midsection arranged adjacent to the resonator, in which the sonotrode is supported in the housing, a rear end coupled to the resonator and a front end which is arranged opposite the resonator and is reduced compared to the midsection, the front end being operatively connected to the tool, and wherein a suctioning duct extends through the sonotrode from the front end thereof with to the rear end thereof with the rear end being connected to the resonator.

24. The surgical instrument according to claim 23, wherein the front end of the sonotrode comprises another metal material than aluminum or an aluminum alloy and in particular comprises titanium or iron or steel or an alloy comprising one of these metals, or wherein the front end of the sonotrode is made of another metal material than aluminum or an aluminum alloy and is made in particular of titanium or iron or steel or an alloy comprising one of these metals.

25. The surgical instrument according to claim 20, wherein the resonator is de-signed as a hollow cylinder and that the sonotrode has a rear end mechanically coupled to the resonator and extending through the resonator, said rear end is in mechanical engagement, in particular in threaded engagement with the resonator for the purpose of biasing the piezoceramic element arranged between the resonator and the sonotrode.

26. The surgical instrument according to claim 20, wherein the sonotrode at its front end operatively connected to the tool extends through an opening of the housing with a play, and that a holder element can be set, in particular plugged or screwed, on the housing at its opening, wherein an elastic sleeve extends from the holder element and surrounds the tool at its outer side.

27. The surgical instrument according to claim 20, wherein the tool is a hollow needle.

28. The surgical instrument according to claim 20, wherein the housing comprises plastic material and/or is made of plastic material.

29. The surgical instrument according to claim 20, wherein the housing comprises an inner housing part surrounding at least a portion of the sonotrode and an outer housing part between which an annular space is provided for supplying and/or for directing rinsing fluid to the tool.

30. The surgical instrument according to claim 20, wherein the inner housing part comprises cooling openings within which the sonotrode and in particular the midsection of the sonotrode is exposed and comes in contact with rinsing fluid for the purpose of cooling.

31. The surgical instrument according to claim 28, wherein the inner housing part comprises an exit opening for the front end of the sonotrode, and the outer housing part comprises an exit opening aligned with the exit opening of the inner housing part and spaced from the same in the longitudinal direction of the sonotrode, in which exit opening the front end of the sonotrode is arranged and/or beyond which the front end of the sonotrode protrudes and on which a holder element can be set, in particular plugged or screwed, from which an elastic sleeve extends from the holder element and surrounds the tool at its outer side.

32. The surgical instrument according to claim 31, wherein the annular space between the inner housing part and the outer housing part is adjoined at the exit opening of the inner housing part by a further annular space which is formed by the outer housing part and the front end of the sonotrode and which extends into an interspace defined by a play between the front end of the sonotrode and the exit opening of the outer housing part and, from there, transitions into an interspace between the tool and the elastic sleeve.

33. The surgical instrument according to claim 28, wherein the sonotrode comprises two axially spaced receiving grooves, each for one bearing ring, in particular for an elastic bearing ring and preferably an O-Ring, both bearing rings abutting on the housing from inside and in particular on the inner housing part for the purpose of supporting the ultrasonic transducer.

34. The surgical instrument according to claim 28, wherein the sonotrode comprises a receiving groove for a bearing ring, in particular an elastic bearing ring and preferably an O-ring, at its rear end opposite the front end or in its central portion, and wherein the front end of the sonotrode is centered by inwardly protruding ribs or the like protrusions of a connecting piece of the housing.

35. The surgical instrument according to claim 30, wherein the cooling openings are arranged in the region of the housing or the inner housing part situated between the two bearing rings and the two receiving grooves are formed in the midsection of the sonotrode.

36. The surgical instrument according to claim 28, wherein the housing comprises an inlet opening in fluid communication with the annular space for the supply of rinsing fluid.

37. The surgical instrument according to claim 28, wherein the housing has a passage opening for a suctioning hose at its rear end opposite the front end of the sonotrode, which hose is connected to the suctioning duct extending through the sonotrode.

38. The surgical instrument according to claim 28, wherein a connection cable connected to the piezoceramic element extends out from the housing.
